(19) United States
(12) Patent Application Publication
(10) Pub. No.: US 2004/0132027 A1
(43) Pub. Date: Jul. 8, 2004

(54) Screening method for discovering auxiliary agents promoting endocytosis

(76) Inventors: Christine Lang, Berlin (DE); Reinhard Gessner, Berlin (DE); Birgit Neukamm, Berlin (DE); Bianka Prinz, Berlin (DE); Elke Nevoight, Berlin (DE)

Correspondence Address: Mark D Wieczorek, P O Box 70072, San Diego, CA, 92167, US

(21) Appl. No.: 10/362,794

(22) PCT Filed: Jun. 30, 2001
(86) PCT No.: PCT/EP01/07509

(30) Foreign Application Priority Data
Aug. 23, 2000 (DE) 100 41 411.7

(51) Int. Cl. C12Q 1/68; C12Q 1/04; C12N 1/18

(52) U.S. Cl. 435/6; 435/34; 435/254.2

(57) ABSTRACT

The invention relates to a screening method for discovering auxiliary agents promoting endocytosis of low molecular and/or macro-molecular compounds, especially nucleic acids, in cells, comprising the following steps: a) a yeast strain is selected and yeast cells are cultivated therefrom, b) the yeast cells are incubated with a potential auxiliary agent promoting the endocytosis of low molecular and/or macro-molecular compounds, especially nucleic acids, in cells in addition to a nucleic acid which is replicable and/or replicable in the nucleus and/or in the cytosol of the yeast cells or a low and/or macro-molecular compound, c) the transformation rates or the transport rates into the cytosol or the nucleus are determined and analysis occurs if the transformation rates or transport rates increase in comparison with the transformation rates or transport rates in a reference test in which the auxiliary substance is not used, d) the auxiliary substance is selected or rejected according to the result of the value obtained for the increase in the transformation rates or transport rates in step c), a screening method of the above-mentioned variety, wherein vertebrate cells are used, in addition to the use of auxiliary agents obtained according to such method in order to produce pharmaceutical substances.

### FIELD OF THE INVENTION

The invention relates to a screening method for discovering auxiliary agents promoting endocytosis of low molecular and/or macro-molecular compounds, especially nucleic acids, in cells and to the use of auxiliary agents obtained according to such a screening method for the production of pharmaceutical substances.

The term endocytosis designates the reception of extracellular, corpuscular or dissolved, in most cases macro-molecular material in a cell. Macro-molecular are compounds having a molecular weight above 300 Da, preferably above 500 Da, most preferably above 1,000 Da. To the naturally uptakable macro-molecules belong for instance antigen-antibody complexes, lipoproteins, LDL, transferrin and nucleic acids. Naturally uptakable corpuscular material includes for instance viruses, bacteria and protozoa.

For instance in connection with gene therapeutic measures, but also for using directly translatable RNA molecules or nucleic acids acting as an antisense RNA, ribozymes or RNA aptamers (inclusive non-natural derivatives such as PNA), the macro-molecular nucleic acids have to be taken up, at least as a first step, in the cytosol, possibly also transported on into the nucleus. Methods and auxiliary agents, such as the low molecular active substances being favored up to now in the pharmaceutical industry are not suitable here. Rather, the natural endocytosis mechanisms have to be used, and the transport rates from the cell membrane to the active location have to be increased to useful levels by suitable auxiliary means.

Auxiliary agents are substances which act on one or more steps of the endocytosis increasing the transport rate or which modulate the latter.

### BACKGROUND OF THE INVENTION AND PRIOR ART

Gene therapy is a promising method for healing a multitude of diseases caused by a germline or somatic gene defect, such as hereditary diseases and cancer (Crit Rev Ther Drug Carrier Syst 1999; 16(2): 147-207). In gene therapy, nucleic acids are brought into the target cell in order to directly remedy there the defect. In the last decades, several methods for the introduction of nucleic acids in the cell have been developed. Hereto belongs the introduction of nucleic acids in cells by means of viral vectors, lipofection and the directed reception by means of receptor-mediated endocytosis, such as for instance the transferrin reaction. The drawbacks of viral systems compared to the remaining systems have become more and more significant in the past years, so that the future of the gene therapy will probably rely on the use of the other systems, e.g. the transferrin reaction. This is a method based on the natural endocytosis mechanisms of the cell. The transferrin reaction uses the natural transferrin-transferrin receptor-endocytosis system securing the iron supply in proliferating, differentiating and hemoglobin synthesizing human cells (Theil, E. C., Aisen, P. (1987) The storage and transport of iron in animal cells. Iron transport in Microbes, Plants, Animals, pp. 491-520, Winkelmann, G., van der Helm, D. and Neilands, J. B. (editors), VCH, Weinheim). In the transferrin reaction, the naturally existing transferrin-transferrin receptor-endocytosis system was modified by coupling the DNA to the ligand transferrin (E. Wagner et al., 1991, Bioconjugate Chem. 2:226-231). The ligand specifically binds to the cell surface receptor of the target cell and is taken up by endocytosis. In order to neutralize the negative charge of the DNA, to condense the DNA and to thus make it accessible for an uptake, polycations such as polyethyleneimine or polylysine are employed. It could furthermore be shown that even condensed DNA not coupled to a ligand is endocytically taken up (W. T. Godbey et al., 1996, PNAS 06:5177-5181), and this system is described as polyfection. For a gene therapeutic application of the systems developed up to now for the well-aimed introduction of nucleic acids in eukaryontic cells by endocytosis, the uptake rate, stabilization of the DNA prior to degradation in the cellular compartments and transfer efficiency at the active position (cytosol or nucleus) is however insufficient (Mahato R I (1999) Non-viral peptide-based approaches to gene delivery. J Drug Target. 7(4):249-68).

In addition to gene therapy, there are other treatment approaches and use of in most cases macro-molecular nucleic acids. A first approach is the introduction of directly translatable RNA in a target cell, then an active substance coded by the RNA being expressed by the cellular mechanisms. By means of anti-sense RNA, RNA aptamers and ribozymes, processes on RNA level naturally occurring in a cell because of defects, mutations etc. can be modulated. Also in this context, the use and modulation or promotion of natural endocytosis processes is required, in order that the nucleic acid is introduced in a sufficient amount in the cell.

Equivalent considerations apply to other low and/or macro-molecular molecules in therapeutic connections, which can only difficultly or not at all overcome the membrane barrier and can be received in the complex with proteins, nucleic acids or synthetic macro-molecules by means of endocytosis in the cell. Hereto belong in particular medicines existing in an ionized form in the plasma and for which there is no suitable cellular transport system. Furthermore it is desirable to have available a method for the uptake of lipophilic pharmaceuticals having a high protein binding and being able to be taken up, after in vitro binding to a suitable carrier protein or another suitable macro-molecule, to be taken up by endocytosis in the target cell.

### TECHNICAL OBJECT OF THE INVENTION

The invention is based on the technical object to improve the transport rate from the endosomal vesicles to the target location, the uptake rate, the stabilization and the transfer efficiency of low and/or macro-molecular compounds, in particular of nucleic acids, in methods using the natural endocytosis mechanisms for the introduction of macro-molecular compounds, in particular nucleic acids, in cells. The invention is based on the further object to specify a method for discovering auxiliary agents by means of which the above improvements can be achieved.

### BASICS OF THE INVENTION

For achieving this technical object, the invention teaches a screening method for discovering auxiliary agents promoting the endocytosis of low molecular and/or macro-molecular compounds, especially nucleic acids, in cells, comprising the following steps: a) a yeast strain is selected and yeast cells are cultivated therefrom, b) the yeast cells are incubated with a potential auxiliary agent promoting the endocytosis of low molecular and/or macro-molecular compounds, especially nucleic acids, in cells in addition to a nucleic acid which is replicable and/or a low and/or macro-molecular compound, especially nucleic acid, detectable in the nucleus and/or in the cytosol, c) the transformation rates or the transport rates into the cytosol or the nucleus are determined and analysis occurs if the transformation rates or transport rates increase in comparison with the transformation rates or transport rates in a reference test in which the auxiliary substance is not used, d) the auxiliary substance is selected or rejected according to the result of the value obtained for the increase in the transformation rates in step c). The term endocytosis comprises for the purpose of this invention also the transport of compounds to their active location in a target cell. Nucleic acids may be bound or coupled to ligands. The ligand is taken up in the unmodified or modified state by receptor mediated endocytosis and leads to the stable transformation of the yeast cell (see e.g. Neves C., Escriou V., Byk G., Scherman D., Wils P., Cell Biol. Toxicol. (1999); 15(3):193-202 or Neves C., Byk G., Scherman D., Wils P., FEBS Letters (1999) 453:41-45). It is understood that for the purpose of the invention, several auxiliary agents may also be used at the same time, either low molecular or macro-molecular or in mixtures thereof.

The reference test is preferably performed by replacing the step b) by the following step: b′) the yeast cells are incubated with a nucleic acid which is replicable and/or a macro-molecular compound, especially a nucleic acid, detectable in the nucleus and/or in the cytosol of the yeast cells, with otherwise unchanged further method steps.

With regard to a testing of selective auxiliary agents for use in human cells it is recommendable to add the following step: e) a potential auxiliary agent selected in step d) is subjected to a determination of the increase of the transformation rates or transport rates in comparison with the transformation rates or transport rates without using the auxiliary agent for vertebrate cells and the auxiliary agent is selected or rejected according to the increase in the transformation rates or transport rates.

Particularly preferred is an embodiment of the invention, wherein parallely or successively the increase in the transformation rates or transport rates for one and the same potential auxiliary agent is performed with wild-type and mutated yeast cells.

In a variant of the invention, a screening method for discovering auxiliary agents promoting the endocytosis of nucleic acids in cells is suggested, said method comprising the following steps: a) a vertebrate cell strain is selected, and cells are cultivated herefrom, b) the cells are incubated with a potential auxiliary agent promoting the endocytosis of nucleic acids in cells and with DNA transcribable in the vertebrate cells and/or with a directly or indirectly detectable RNA, c) the cells of step b) are examined for the total amount of taken up or adsorbed nucleic acid, the amount of intracellular nucleic acid, the amount of nucleic acid in the nucleus, the transfection rate and/or the amount of gene product, compared with a reference test in which the auxiliary substance is not used, d) the auxiliary substance is selected or rejected according to the results of step c).

Finally, the invention teaches the use of an auxiliary agent, obtainable by a method according to the invention, for the production of a pharmaceutical substance for promoting the uptake of macro-molecular compounds, in particular nucleic acids, in cells.

The invention is based on the finding that for the screening of substances leading to an increase of the endocytic uptake of macro-molecular compounds, in particular nucleic acids, into the target cell and the translocation thereof to the active location, yeast, in particular the organism Saccharomyces cerevisiae, will form a particularly well suited model system. The basic mechanisms of the uptake and transport seem to be rather conserved between the various eukaryontic organisms. The intracellular transport processes for yeast are very well researched. For the yeast Saccharomyces cerevisiae it has been observed that receptors localized in the plasma membrane, such as the alpha-factor receptor, are endocytized and transported via the endosomes to the vacuole where they are decomposed (Schandel, K., Jenness, D. (1994) Direct evidence for ligand-induced internalization of the yeast alpha-factor pheromone receptor. Mol Cell. Biol., 14:7245-7255; Riballo, E., Herweijer, M., Wolf, D., Lagunas, R. (1995) Catabolit inactivation of the yeast maltose transporter occurs in the vacuole after internalization by endocytosis. J. Bacteriology, 177:5622-5627). A “recycling”wherein the receptor after uptake in the cell by endocytosis is transported from the endosomes back to the plasma membrane, has not yet been described for yeast, other than for mammal cells. Mammal cells bring for instance the transferrin receptor/transferrin complex by a “recycling”from the endosomes back into the plasma membrane. In mammal cells there are however clear hints that modified transferrin and transferrin-DNA complexes are not “recycled”, but degraded in the lysosomes (Wagner, E., Curiel, D., Cotton, M. (1994) Delivery of drugs, proteins and genes into cells using transferrin as a ligand for receptor-induced endocytosis, Advanced Drug Delivery Reviews, 14:113-135). Therefore the yeast, in particular Saccharomyces cerevisiae, is very well suited as a model for the uptake of low and/or macro-molecular compounds, for instance nucleic acids or nucleotides, in the human cell by endocytosis. It can therefore be assumed that at least a large fraction of the highly efficient auxiliary agents found with the invention will show a high uptake rate, a fair stabilization and a high transfer efficiency in the human system, too.

The invention is based on the further finding that with yeast cells, other than for mammal cells, a multitude of defect mutants can be produced in a short time, wherein defined genes of the endocytosis pathway are interrupted in a well-aimed manner. The application of the mutants makes it possible to experimentally separate the individual steps of the endocytosis pathway from the plasma membrane through the endocytic vesicles to the vacuole and to determine their influence on the transfer into the cytosol or the nucleus of the cell. Based on the findings obtained therefrom, the substances can be tested for whether they can break through the found bottlenecks at the uptake and translocation from the endocytic vesicles to the target location. In other words, the invention may also be used for determining the functional mechanisms of individual auxiliary agents. Since the process of the uptake of macro-molecular compounds or of nucleic acids is a multi-step one, in this way also auxiliary agent combinations can be determined, which overall achieve the desired effect. With such auxiliary agent combinations, the uptake rate and transfer efficiency are considerably increased, since all bottle-necks are affected in a well-aimed and specific manner.

Yeast cells have, compared to mammal cells, further the advantage of a much simpler handling. They can be cultivated in a simple manner and in large cell quantities, and a large quantity of mutants or transformants can parallely be analyzed. Because of the properties mentioned above, yeasts are well suited for experiments requiring a miniaturization, because of the large quantities of parallel sections. Compared to human systems, as a result, thus a very high throughput is achieved with screening.

The transport rate can be determined by a time-dependent, PCR-supported measurement of the amount of nucleic acid in the various cellular compartments (complete cells, DNase digested complete cells, cell nuclei) and of its biological activity (GFP, luciferase measurement). With a precise knowledge of the absolute amounts, the transport rate can exactly be determined. For the measurement of the influence of an auxiliary agent on individual steps of the transport, however usually a simple comparison of the amount of nucleic acids in the various compartments in presence and absence of the auxiliary agent is sufficient.

The transport rate can also be defined by the transformation rate, measured according to example 1. Corresponding comparative measurements with regard to various intracellular target compartments can easily be developed. Measurements are always taken therein with regard to a reference test under otherwise identical test conditions. Alternatively, comparisons can be made to the use of a defined quantity or concentration of chloroquine as a standard.

For the purpose of the invention, it is also possible to examine the influence of other conditions, such as temperature, pressure, electric fields, electromagnetic alternating fields, current and properties such as composition of the medium.

### EMBODIMENTS OF THE INVENTION

With regard to an examination being efficient and working at a high throughput capacity, it is preferred that a multitude of different potential auxiliary agents promoting the endocytosis of macro-molecular compounds, in particular nucleic acids, in cells are parallely examined for the increase of the transformation rates or of the transport rates. This can advantageously be made with yeast cells, since they are considerably easier to handle than mammal cells and can be cultivated simply and in large quantities. In addition, a multitude of mutants can simultaneously and easily be examined, thus information about the active locations being quickly obtainable. Further, the easy handling permits to also examine any auxiliary agent combinations effectively and with comparatively low time consumption.

Preferably, the yeast cell strain is selected from the group comprising “RPY10, 4 STLU, AH22, DBY747, GRF18, CBS6503, ABYS86, MF9, YMTA, BJ3505, GAG2”, in particular RPY10. When using RPY10, it is preferred that mutated yeast cell strains are selected from the group comprising “deletion mutants of the strain RPY10 with deletion of the of the genes YPT51, YPT7, VPS27 and/or PEP4”. Generally, mutant strains can be used which interrupt the transport of endocytized material at defined locations of the endocytosis pathway, or which lack specific DNA/RNA degrading enzymes. These deletion mutants are designed from the original strains, so that they only differ in the deletion of the gene being relevant for the endocytic transport (see for instance Guldener, U., Heck, S., Fiedler, T., Beinhauer, J., Hegemann, J H., A new efficient gene disruption cassette for repeated use in budding yeast. Nucleic Acids Research, 1996, 24 (13): 2519-2524). It may also be possible to use temperature-sensitive strains forming a defect at a temperature above the optimum growth temperature.

In principle, any cell lines, primary cells or tissue explants can be used in the variant using vertebrate cells. Preferably mammal cell lines, ideally human cell lines are used. It is suitable that the vertebrate cell line is selected from the group comprising “HepG2, K562, B16, MeWo, CMT-93, RMA, LL/2, 293, HT-1080, COS-1, COS-2, CV-1, CHO, OCM, HL-60, L929, 3T3, BLK CL.4, HeLa, T84”, in particular HepG2.

In an embodiment of the invention with a determination of the transfection rate and/or the amount of gene product, the nucleic acid may include a marker gene, preferably a luciferase gene and/or EGFP, and wherein is determined the increase of the transfection rate by counting the cells expressing the marker gene or the luciferase activity. When determining the transport rates of macro-molecular compounds, also of nucleic acids, generally other methods may also be used, for instance radioactive markers, fluorophores or chemically modified compounds permitting a secondary qualitative and/or quantitative proof. For nucleic acids, RNA molecules being directly translatable and coding for a detectable protein, or RNA molecules having an otherwise detectable function the cell, such as for instance antisense RNA, ribozymes or RNA aptamers, may also be used.

With regard to an evaluation, it is preferred that the transformation rates or transport rates are referred to the number of divisible cells of step b) by determining the number of colony-forming units after cultivation of the cells of step b) in a selection medium adapted to the nucleic acid and division by the number of the colony-forming units after cultivation of the cells of step b) in full medium. A successful uptake of for instance double-stranded circular DNA will lead to an expression of a marker gene for which the yeast strain is auxotrophic. A quantification takes place by the number of colony-forming units on a selection medium, referred to the number of divisible cells, in order to take account of a potential toxicity for the cells of an auxiliary agent to be tested.

The verification of the successfully transformed or transfected cells and the determination of the distribution of the nucleic acids in different intracellular compartments, and thus the respective transformation rates or transport rates can be performed by means of PCR.

In the following, the invention is described in more detail, based on examples of execution.

### EXAMPLE 1

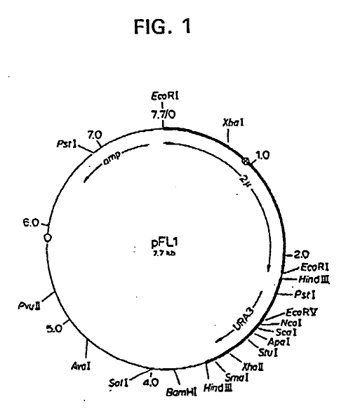

A test method for the quantification of the uptake and the endocytosis transfer was made as follows. 1×10 cells from the logarithmic growth phase of the strain RPY10 (Matα, leu2-3, ura3, his4 ade6 (4×10⁷−7×10⁷ cells per ml) (Piper, R. C., Whitters, E. A., Stevens, T. H. (1994) Yeast VpsS4p is a Sec 1p-like protein required for the consumption of vacuolar targeted, post-Golgi vesicles. Eur. J. Cell Biol. 65:305-318) were incubated for 24 h with 10 μg of the purified plasmid YEP24 (see: D. Bothstein et al., 1979, Gene 8:17-24), with and without auxiliary agent. The structure of the plasmid is shown in FIG. 1.

Prior to the incubation, the cells were washed with the plasmid with at least twice the same volume of H₂O and taken up in 1 ml 1M saccharose (pH 3.3, high concentrations of fermentable sugar), re-suspended with the pipette tip and brought in one 10 ml each falcon tube. Immediately after this step, 5 μl chloroquine as auxiliary agent (strain solution 100 mM) were pipetted thereto in 1 tube and not in a second one. Both were incubated for 5 min at room temperature.

Then 10 μg DNA from the same DNA preparation were pipetted thereto (in 3-100 μl). The falcon tubes were firmly closed and agitated for 24 hours in a water bath or breeding room at 24° C. (the optimum transformation rate is at 24° C.-28° C). For practical reasons, incubation over night is also possible.

After the incubation, 200-500 cells per plate on YE plates (full medium) were plated out, and the colony-forming units (CFU) were determined. The remaining cells were plated out on 3-5 plates with selection medium. After 5 days at 24° C., the CFU were counted and tested by means of PCR.

For the evaluation, the transformants per used cells were referred to the number of divisible cells after the incubation. There did result values for the transformation rate of 10.2 [10⁻⁸ transformants/CFU] with chloroquine and 0.8 [10⁻⁸ transformants/CFU] without chloroquine.

### EXAMPLE 2

An examination with mutant strains was performed as follows. 1×10⁹ cells from the logarithmic growth phase of the strain RPY10 (Matα, leu2-3, ura3, his4, ade6) (approx. 5×10⁷ cells per ml) and deletion mutants of the strain with deletion of the genes:

YPT51 (Singer-Krüger B., Stenmark H., Düsterhöft, A., Phillipsen, P., Yoo, J.-S., Gallwitz, D., Zerial, M. (1994) Role of the three rab5-like GTPases, ypt51p, yptslp, and ypt53p, in the endocytic and vacuolar protein sorting pathway of yeast. JCB, 125(2):283-298)

YPT7 (Wichmann, H. et al., Hengst, L., Gallwitz, D. (1992) Endocytosis in yeast. Evidence for the involvement of a small GTP-binding protein (Ypt7 p). Cell 71(7):1131-42

VPS27 (Piper, R. C., Cooper, A. A., Yang, H., Stevens, T. H. (1995) Vps27 controls vacuolar and endocytic traffic through a prevacuolar compartment in Saccharomyces cerevisiae. JCB 131 (3) 503-617

PEP4 (Meussdoerffer, F., Tortora, P., Holzer, H. (1980) Purification and properties of proteinase A from yeast. J. Biol. Chem, 255 (24):12087-93) In this case the strain SF838-9D, the pep4-3 mutant of the strain RPY10 (Piper et al. (1995)) SF838-9D with a ypt7 deletion or another combination of two defects in a strain

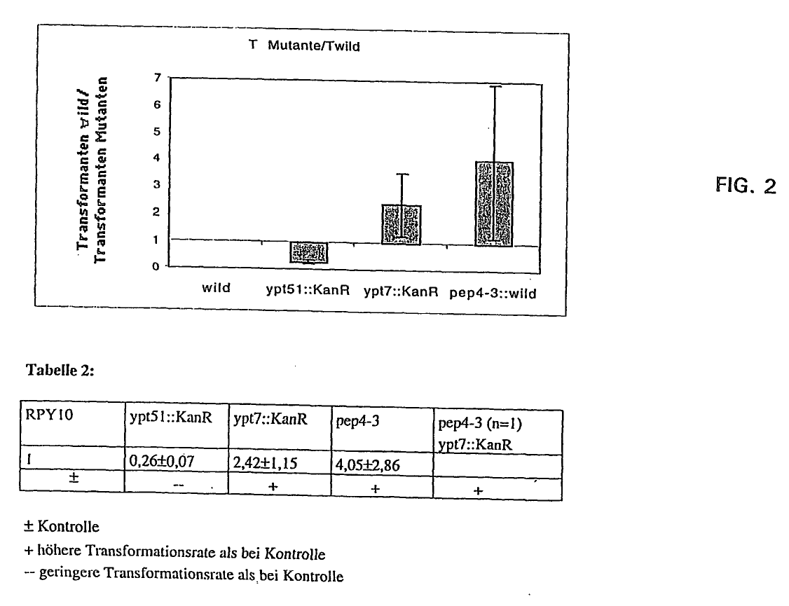

were treated as in example 1. For the evaluation, all transformation rates were referred to the strain RPY10 having no defect in the genes of the endocytosis pathway. The results (n=3) are shown in FIG. 2 and in Table 2.

On the whole, the transport flow of endocytically taken-up substances in yeast by the mutants is affected as follows, in the basic simplified model for the transport steps for the liquid phase endocytosis and the receptor-mediated endocytosis, a large degree of correspondence being assumed:

In the wild type the macro-molecules to be endocytized will first collect at the cell membrane, possibly bind to specific receptors, and accumulate in certain regions. Then follows an internalization, i.e. the uptake in the cell by plasma membrane invaginations, and extracellular liquid and substances dissolved therein are also taken up. After successful internalization, the endocytized molecules appear in the peripheral endosomes which lie immediately underneath the plasma membrane. From there they will pass into the perinuclear endosomes being close to the nucleus. The interior of the endosome compartment is slightly acidic (ATP-driven proton pump), generally internal endosomes being more acidic than peripheral ones. From the endosomes, endocytized molecules pass into a prevacuolar compartment, which is also supplied with transport vesicles from the Golgi apparatus transporting the newly synthesized proteins intended for the vacuoles. From the prevacuolar compartment, the endocytized complexes move into the vacuole, where they are degraded.

The internalization can be blocked by the temperature-sensitive mutants end 3 and end 4 at the permissive temperature. This blocking is described in the literature for the alpha-factor/alpha-factor receptor complex as well as for the liquid phase endocytosis marker Lucifer Yellow. The blocking of this step can prevent an uptake of nucleic acids by endocytosis and serve as a check for the mechanism of the uptake.

The transport between the endosomes and the vacuole is blocked by the deletion of the genes YPT51, YPT7 and VPS27. This will lead to a transport pile-up at selected key positions of the endocytosis pathway. The yeast proteins ypt51p and ypt7p show a high homology to the mammal proteins rab5and rab7, membrane-associated GTPases having key roles in the endocytosis pathway of mammal cells. rab5has a regulatory effect in the early steps of the transport between the plasma membrane and the endosomes, and rab7in the late steps of the endocytosis pathway (Chavrier, P., Parton, R. G., Hauri, H.-P., Simons, K., Zerial, M. (1990) Localization of low molecular weight GTP binding proteins to exocytic and endocytic compartments. Cell 62:317-329, Bucci). For yeast cells which do not express the protein ypt51p, a reduced acidification of the vacuole is described. In the YPT7deletion strain, further a delayed maturing of the soluble vacuolar enzymes is described. The deletion of the gene VPS27leads to a blocking of the endocytosis pathway behind the prevacuolar compartment, which will lead there to a strong accumulation of Golgi proteins and endocytized material in this compartment. By the proteinase A being coded by the gene pep4 and localized in the vacuole, certain degradation enzymes are proteolyzed at their arrival in the vacuole I in such a manner that they will only then develop their activity. The lack of the proteinase A will thus lead to a reduced hydrolysis in the vacuole.

### EXAMPLE 3

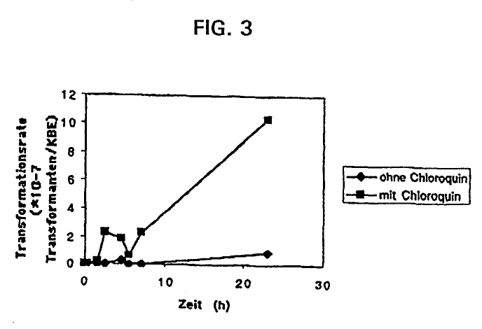

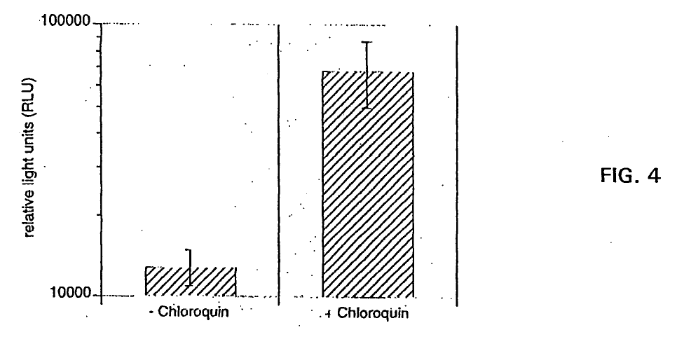

Procedure same as in example 1, however the incubation time was varied in several experiments between 0.5 and 24 h. The results of the measured transformation rates, with and without chloroquine, in dependence of the incubation time are shown in FIG. 3.

### EXAMPLE 4

1×10⁹ cells from the logarithmic growth phase of the strain RH144-3D (Mata, leu2, ura3, his4) (4×10⁷ -7×10⁷ cells per ml) (Raths, S., Rohrer, J., Crausaz, F., Riezmann, H., 1993, JBC, vol. 120(1), 55-65) were incubated with 10 μg of the purified plasmid YEP24 (see: D. Botstein et al., 1979, Gene 8:17-24) for approx. 4 h.

Prior to the incubation with the plasmid, the cells were washed with at least twice the same volume of H₂O and taken up in 1 ml 1M saccharose (pH 3.3, high concentrations of fermentable sugar), re-suspended with the pipette tip and brought in one 10 ml each falcon tube. Immediately after this step, 5 μl chloroquine as auxiliary agent (strain solution 100 mM) were pipetted thereto in all tubes. All tubes were incubated for 5 min at room temperature.

After the incubation, alpha-factor in a final concentration of 1 μM and 10 μg and as a check into a second tube only 10 μg from the same DNA preparation were pipetted thereto (in 3-100 μl). The falcon tubes were firmly closed and agitated for 2.5 hours in a water bath or breeding room at 24° C.

After the incubation, 200 -500 cells per plate on YE plates (full medium) were plated out, and the colony-forming units (CFU) were determined. The remaining cells were plated out on 3-5 plates with selection medium. After 5 days at 24° C., the CFU were counted and tested by means of PCR.

The results for 1 μM alpha-factor final concentration were a value of 1.56×10⁻⁷ transformations/CFU and without alpha-factor a value of 0.8×10⁻⁷ transformations/CFU as the transformation rate.

The alpha-factor/alpha-factor receptor complex is endocytically taken up and degraded in the vacuole. The alpha-factor can on the one hand as a ligand be so modified that (macro-) molecules can be coupled thereto, without reducing the uptake rate or even increasing the latter. On the other hand it is possible to strengthen the uptake rate of DNA not coupled to the ligand by addition of the ligand and the simultaneous induction of the endocytosis process.

### EXAMPLE 5

The human cell line HepG2 was transfected by using the commercially available transferrin infection system by means of directed endocytosis according to instructions with a vector which contains cDNA for EGFP as well as for the firefly luciferase, each under supervision of a constitutively active viral promoter.

The transfected cells were washed 3 times with PBS 48 h after the transfection, incubated for 2 min with 0.25% trypsin in PBS, removed from the cell culture dish and provided with fresh medium with 10% FCS (fetal calf serum). Aliquots of the cells were investigated as follows:

a) The amount of the total DNA (surface-bound and intracellular) was determined by DNA extraction from 10⁵ cells and subsequent quantitative PCR using a defined quantity of the same purified plasmid as a reference sample.

b) Further 10⁵ cells were treated for 30 min at 37° C. with 1 U/μl DNase I in 50 μl TBS/Mg (25 mM Tris-HCl, pH 7.5, 137 mM NaCl, 2.7 mM KCl, 10 mM MgCl₂), washed in PBS and then examined as in a).

c) Further 10⁵ cells were treated with DNase I as described in b), however after the last washing they were not subjected to a DNA isolation, but their cell nuclei were first isolated, as described in the document Dignam et al., Methods Enzymol., 101:582-598, 1983, in order to subsequently obtain DNA from them and to determine the latter quantitatively, as described under a)

In a) to c), an additionally made quantitative detection of cellular DNA, for instance by means of quantitative PCR, served for the standardization of the amount of taken-up DNA.

d) Nearly 48 h after the transfection and shortly prior to washing and trypsinizing, the cells were examined under the fluorescence microscope for the relative share of green-fluorescing cells and thus for the qualitative transfection efficiency (transfection rate).

e) Another aliquot of the cells was supplied according to manufacturer's instructions to a quantitative luciferase assay.

All investigations a) to e) were made parallely on cells which have been transfected with or without addition of chloroquine. In this way it can be differentiated whether chloroquine has an influence on the total amount of adsorbed or taken-up DNA (a), on the amount of intracellular DNA (b), on the amount of DNA in the nucleus (c), on the transfection rate (d) or on the amount of gene product (e). By a comparison of the results it can be determined on which level chloroquine increases the transfection efficiency.

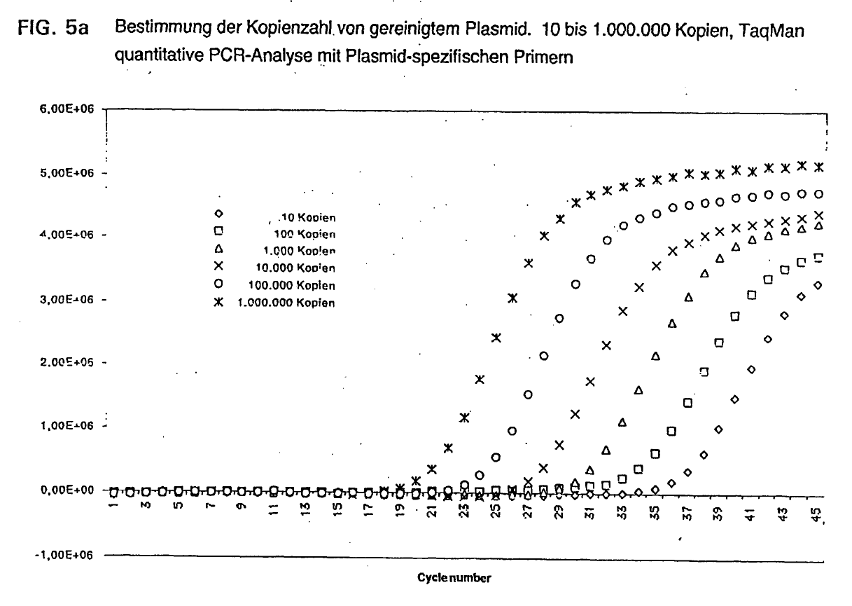

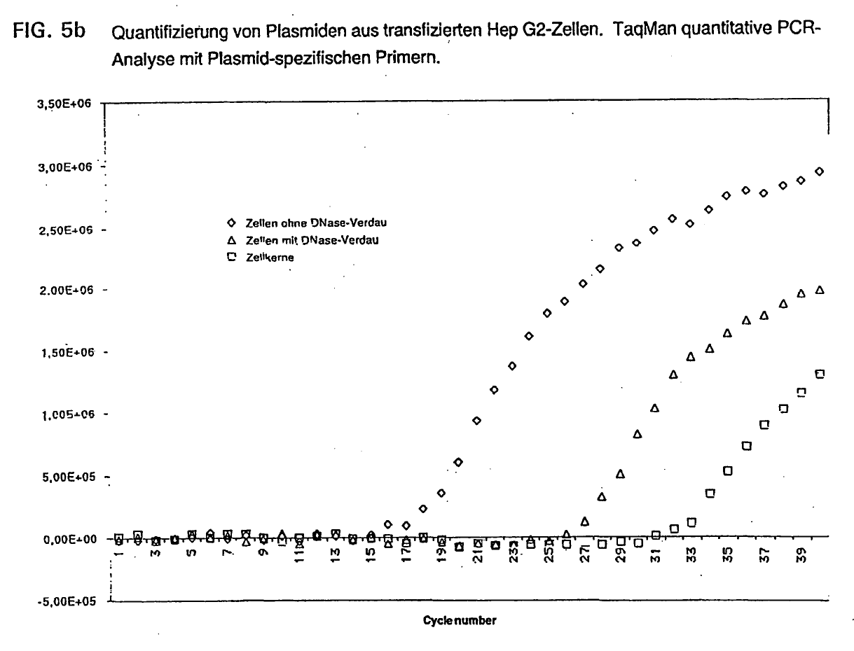

The results are summarized in FIGS. 4 and 5a, b. In FIG. 4 is shown the influence of chloroquine on the amount of expressible cellularly taken-up vector DNA by measurement of the luciferase activity. With chloroquine, a nearly 10 times higher activity was measured. In FIG. 5, the determination of the DNA amount in different cellular compartments by means of quantitative PCR is summarized. FIG. 5a is a calibration curve. In FIG. 5b can be seen the amounts of DNA in different regions of the cells. The rhombuses show values from step a), the triangles from step b) and the squares from step c).

1. A screening method for discovering auxiliary agents promoting endocytosis of low molecular and/or macro-molecular compounds, especially nucleic acids, in cells, comprising the following steps:
a) a yeast strain is selected and yeast cells are cultivated therefrom,
b) the yeast cells are incubated with a potential auxiliary agent promoting the endocytosis of low molecular and/or macro-molecular compounds, especially nucleic acids, in cells in addition to a nucleic acid which is replicable and/or detectable in the nucleus and/or in the cytosol of the yeast cells or low and/or macro-molecular compounds,
c) the transformation rates or the transport rates into the cytosol or the nucleus are determined and examined for an increase of the transformation rates or transport rates, in comparison with the transformation rates or transport rates in a reference test in which the auxiliary substance is not used,
d) the auxiliary substance is selected or rejected according to the result of the value obtained for the increase in the transformation rates in step c).

2. A screening method according to claim 1, wherein the reference test is performed by replacing the step b) by the following step:
b′) the yeast cells are incubated with a nucleic acid which is replicable and/or a low and/or macro-molecular compound or nucleic acid detectable in the nucleus and/or in the cytosol of the yeast cells,
with otherwise unchanged further method steps.

3. A screening method according to claim 1 or 2, wherein the following step is added:
e) a potential auxiliary agent selected in step d) is subjected to a determination of the increase of the transformation rates or transport rates in comparison with the transformation rates or transport rates without using the auxiliary agent for vertebrate cells, and the auxiliary agent is selected or rejected according to the increase in the transformation rates or transport rates.

4. A screening method according to one of claims 1 to 3, wherein a multitude of different potential auxiliary agents promoting the endocytosis of low and/or macro-molecular compounds, in particular nucleic acids, in cells are parallely examined for the increase of the transformation rates or of the transport rates.

5. A screening method according to one of claims 1 to 4, wherein parallely or successively the increase in the transformation rates or transport rates for one and the same potential auxiliary agent is performed with wild-type and mutated yeast cells.

6. A screening method according to one of claims 1 to 5, wherein the yeast cell strain is selected from the group comprising “RPY10, 4STLU, AH22, DBY747, GRF18, CBS6503, ABYS86, MF9, YMTA, BJ3505, GAG2”, in particular RPY10.

7. A screening method according to claim 5 or 6, wherein the mutated yeast cell strain is selected from the group comprising “deletion mutants of the strain RPY10 with deletion of the of the genes YPT51, YPT7, VPS27 and/or PEP4”.

8. A screening method according to one of claims 1 to 7, wherein the vertebrate cell line is selected from the group comprising “HepG2, K562, B16, MeWo, CMT-93, RMA, LL/2, 293, HT-1080, COS-1, COS-2, CV-1, CHO, OCM, HL-60, L929, 3T3, BLK CL.4, HeLa, T84”, in particular HepG2.

9. A screening method according to one of claims 1 to 8, wherein the nucleic acid includes a marker gene, preferably a luciferase gene and/or EGFP, and wherein is determined the increase of the transfection rate by counting the cells expressing the marker gene or the luciferase activity.

10. A screening method according to one of claims 1 to 8, wherein the transformation rates or transport rates are referred to the number of divisible cells of step b) by determining the number of colony-forming units after cultivation of the cells of step b) in a selection medium adapted to the nucleic acid and division by the number of the colony-forming units after cultivation of the cells of step b) in full medium.

11. A screening method according to one of claims 1 to 10, wherein a determination of nucleic acids transported into the cytosol or into the nucleus or possibly integrated therein is performed by means of PCR.

12. A screening method for discovering auxiliary agents promoting the endocytosis of nucleic acids, comprising the following steps:
a) a vertebrate cell strain is selected, and cells are cultivated herefrom,
b) the cells are incubated with a potential auxiliary agent promoting the endocytosis of nucleic acids in cells and with a DNA transcribable in the vertebrate cells and/or with a nucleic acid directly or indirectly detectable in the cytosol,
c) the cells of step b) are examined for the total amount of taken up or adsorbed nucleic acid, the amount of intracellular nucleic acid, the amount of nucleic acid in the nucleus, the transfection rate and/or the amount of gene product, compared with a reference test in which the auxiliary substance is not used,
d) the auxiliary substance is selected or rejected according to the results of step c).

13. The use of an auxiliary agent, obtainable by a method according to one of claims 1 to 12, for the production of a pharmaceutical substance for promoting the uptake of low and/or macro-molecular compounds, in particular nucleic acids, in cells.
